(19) United States
(12) Patent Application Publication
(10) Pub. No.: US 2024/0263133 A1
(43) Pub. Date: Aug. 8, 2024

(54) PROCESS FOR THE ISOLATION AND CULTURE OF STRAINS, THE STRAINS, USE THEREOF, MEDIA FOR CULTURING THEREOF AND A FORM OF SCYTONEMIN

(71) Applicant: UVERA SA, Lódz (PL)

(72) Inventors: Adam KICIAK, Warszawa (PL); Magdalena JANDER, Warszawa (PL)

(73) Assignee: UVERA SA, Lódz (PL)

(21) Appl. No.: 18/564,630

(22) PCT Filed: May 27, 2022
(86) PCT No.: PCT/PL2022/050034

§ 371 Date: Nov. 28, 2023

(30) Foreign Application Priority Data
May 28, 2021 (PL) P.437991

(51) Int. Cl. C12N 1/20; A61K 8/49; A61K 8/99; A61Q 17/04; C07D 209/94; C09B 61/00; C12P 17/16; C12R 1/01

(52) U.S. Cl. CPC C12N 1/205; A61K 8/492; A61K 8/99; A61Q 17/04; C07D 209/94; C09B 61/00; C12P 17/165; A61K 2800/43; C12R 2001/01

(57) ABSTRACT

The object of the invention is a process for the isolation and culture of strains, the strains, use thereof, media for culturing thereof and a form of scytonemin.

The object of the invention is a process for the isolation and culture of strains, the strains, use thereof, media for culturing thereof and a form of scytonemin. The invention is applicable to biotechnological and cosmetic industry.

Standard isolation and culture methods are known (Rippka et al., 1979; Anahas and Muralitharan, 2015; Singh et al., 2014) which involve collecting a biological material from the endolytic microenvironment, for example pores in stone, either directly into a culture medium (BG11) (Singh et al., 2014) or else placing an isolated biological material on plates with 2% agar and the BG11 growth medium for the selection of monoclonal Cyanobacterium (according to Wolk, 1998) , and subsequently transferring the colonies directly into the BG11 liquid medium (Anahas and Muralitharan, 2015). Direct collection of the biological material into the BG11 liquid medium may not be applicable in many cases, because this would require additional purification and isolation of monoclonal bacterial strains. The culture and purification method reported by Anahas and Muralitharan (2015) on plates with agar and BG11 (according to Wolk, 1998) does not achieve the expected results, because Cyanobacteria colonies collected from extremely dry environments proliferate extremely slowly or not at all. It is desirable to provide a culture method that would enable easy proliferation of various Cyanobacteria strains isolated from the environment whose culture in laboratory conditions using methods known in the art is impossible or at least difficult to accomplish. It is expected that application of the method would as a result enable the isolation of new Cyanobacteria strains having uniquely favorable properties, in particular high productivity of pigments, being also natural ultraviolet (UV) radiation filters. The present invention unexpectedly solved the aforementioned problems.

The first object of the invention is a process for the isolation and culture of Cyanobacteria strains, in particular that deposited in Banco Espanol de Algas Universidad de Las Palmas de GC under number BEA_IDA_0068B or BEA_IDA_0075B, characterized in that it comprises:
a) preparation of a growth medium by enriching it in micro- and macronutrients found in natural sandstone originating from Nubian formations the contents with following in mass percentages: 97.6% quartz, 0.4% muscovite-biotite 1.2% apatite and 0.8% other minerals in trace quantities in the amount of 200 g per 1000 mL of an aqueous medium solution having the following composition per 1000 mL of the medium: 1.5 g NaNO₃, 0.04 g K₂HPO₄, 0.075 g MgSO₄×7H₂O, 0.036 g CaCl₂×2H₂O, 6.0 mg citric acid, 6.0 mg ammonium ferric citrate, 1 mg EDTA, 0.02 g Na₂CO₃, 1 mL of the A5 blend of trace metals with the following composition per 1000 mL of the aqueous A5 blend solution: 2.86 g H₃BO₃, 1.81 g MnCl₂×4H₂O, 0.222 g ZnSO₄×7H₂O, 0.39 g Na₂MoO₄×2H₂O, 0.079 g CuSO₄×5H₂O, 49.4 mg Co (NO₃) 2×6H₂O, subsequently stirring the resulting suspension for 24 hours at 25° C. and subsequent 5-hour sedimentation at 25° C. and filtration thereof; whenever the Applicant uses the phrase pure BG11 medium or agar-free BG11 medium or BG11 medium or medium according to Rippka et al. (1979) or medium with the composition of Table 1 or medium whose composition is disclosed in stage a), but without addition of the stone, this is meant to be the medium with the composition listed below in Table 1.

TABLE 1

| | | Ingredient quantity per | |
|---|---|---|---|
| Ingredient | | 1000 mL of the medium | |
| no. | Chemical name | being an aqueous solution | |
| 1 | NaNO₃ | 1.5 | g |
| 2 | K₂HPO₄ | 0.04 | g |
| 3 | MgSO₄ × 7H₂O | 0.075 | g |
| 4 | CaCl₂ × 2H₂O | 0.036 | g |
| 5 | Citric acid | 6.0 | mg |
| 6 | Ammonium ferric citrate | 6.0 | mg |
| 7 | EDTA | 1 | mg |
| 8 | Na₂CO₃ | 0.02 | g |
| 9 | A5 blend of trace metals | 1 | mL |
| | with the following | | |
| | composition per 1000 mL | | |
| | of the aqueous A5 blend | | |
| | solution: | | |
| | 2.86 g H₃BO₃, 1.81 g MnCl₂ × | | |
| | 4H₂O, 0.222 g ZnSO₄ × | | |
| | 7H₂O, 0.39 g Na₂MoO₄ × | | |
| | 2H₂O, 0.079 g CuSO₄ × | | |
| | 5H₂O, 49.4 mg Co(NO₃)₂ × | | |
| | 6H₂O | | |

b) collection of bacteria from the environment;
c) passaging the biological material collected in stage b) in the liquid medium obtained in stage a), i.e., according to Table 1, enriched with stone, with additional agar with ultimate contents between 2% in the beginning and 0.5% by weight in the end, preferably in three intermediate stages of 4 weeks each of the five stages, i.e. two ultimate stages (initial, final) and three intermediate stages, wherein the growth media in the intermediate stages contain the following quantities of additional agar: 1.75%, 1.5%, 1% by weight, respectively, with respect to the medium obtained in stage a);
d) dissolving the culture solution from final stage c), i.e. containing 0.5% agar, in the aqueous medium solution whose composition is disclosed in stage a) but without addition of the stone and incubation at 25° C. for 2 weeks with stirring.

The second object of the invention is a bacterial strain deposited in Banco Espanol de Algas Universidad de Las Palmas de GC under number BEA_IDA_0068B.

The third object of the invention is a bacterial strain deposited in Banco Espanol de Algas Universidad de Las Palmas de GC under number BEA_IDA_0075B.

The fourth object of the invention is the use of the strain of the invention defined as the second object of the invention for the preparation of a pigment with UV absorption properties, in particular scytonemin or derivatives thereof. Equally preferably the use of the invention comprises application of the resulting pigment, in particular scytonemin or derivatives thereof, for the manufacture of cosmetic products, in particular for sunscreens.

The fifth object of the invention is a medium for culturing Cyanobacteria, containing in 1000 mL of the aqueous medium solution 1.5 g NaNO₃, 0.04 g K₂HPO₄, 0.075 g MgSO₄×7H₂O, 0.036 g CaCl₂×2H₂O, 6.0 mg citric acid, 6.0 mg ammonium ferric citrate, 1 mg EDTA, 0.02 g Na₂CO₃, 1 mL of the A5 blend of trace metals with the following composition per 1000 mL of the aqueous A5 blend solution: 2.86 g H₃BO₃, 1.81 g MnCl₂×4H₂O, 0.222 g ZnSO₄×7H₂O, 0.39 g Na₂MoO₄×2H₂O, 0.079 g CuSO₄×5H₂O, 49.4 mg Co (NO₃)₂×6H₂O, characterized in that it contains natural Nubian sandstone with the following contents in mass percentages: 97.6% quartz, 0.4% muscovite-biotite 1.2% apatite and 0.8% other minerals in trace quantities in the amount of 200 g ground stone/1000 mL of the medium. The disclosed medium is suitable for culturing Cyanobacteria which produce pigments, in particular scytonemin.

The sixth object of the invention is scytonemin crystals having at least one property selected from the following:
X-ray powder diffraction spectrum with characteristic peaks at 2 theta angle values of 2.500°, 4.589°, 5.062°, 8.630° and 9.197°,
specific infrared absorption bands at 3345, 3065, 2961, 2926, 1713, 1591, 1516, 1449, 1296, 1175, 1145, 957, 932, 930, 833 [cm⁻¹] in the IR spectrum (KBr),
decomposition temperature in a range between 365° C. and 380.3° C. with a peak at about 380.3° C. in thermogravimetric/differential thermal analysis (heating/cooling rate: 15/20° C./min),
1H NMR spectrum recorded in pyridine-d5 containing signals at δ 8.98 ppm, 7.99 ppm, 7.86 ppm, 7.75 ppm, 7.48 ppm, 7.33 ppm and 7.22 ppm.

Owing to the invention, it was possible to effectively provide a culture method that enabled easy proliferation of a desirable Cyanobacteria strain isolated from the natural environment whose culture in laboratory conditions using methods known in the art was impossible or at least difficult to accomplish. Owing to the use of the process of the invention, a specific Cyanobacterium strain could be isolated having extremely favorable properties, in particular high productivity for scytonemin of Formula 1, in particular its oxidized form, i.e., at least 1.5% per dry weight of Cyanobacterium.

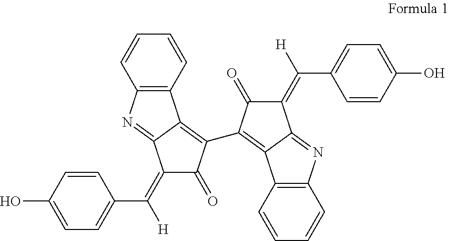 [SMILES: O=C1C(C2=C3C(=Nc4ccccc43)/C(=C\c3ccc(O)cc3)C2=O)=C2C(=Nc3ccccc32)/C1=C\c1ccc(O)cc1]

The taxonomic characteristics of the Cyanobacterium strain were determined based on optical microscopy analysis and on the latest guidelines published in Komerek et al. (2014) and the literature reports found in that paper. Micromorphological characteristics of the test strain show it belongs to the family Chroococcidiopsidaceae (Komerek et al., 2014) and the genus Chroococcidiopsis (Geitler, 1932).

Micromorphological description of the resulting strains:

Cyanobacterium cells obtained in a culture of the invention had the following features:
a) color: blue-green, yellow to light brown;
b) form: single and spherical cells with a diameter of between 1.5 and 5 μm, clustered in colonies—from several to less than twenty cells or else forming aggregates, typically surrounded by a distinct sheath;
c) division: cells divide along two or more planes. After division cell coats typically extend and include daughter cells, which is seen as layering of a colony sheath;
d) thylakoid arrangement: arranged circularly near the cell wall.

Embodiments of the invention are shown in the drawings, wherein

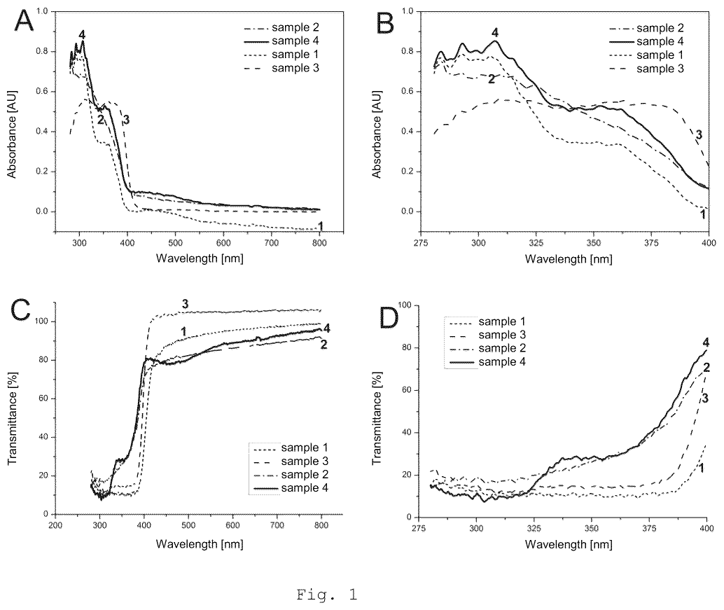

FIG. 1 shows comparative results of absorbance (A and B) and transmittance (C and D) tests for selected commercially available creams with SPF 30 and 50 (samples 1-3) and samples (no. 4) with scytonemin added, wherein the tests were performed using a thin-layer material to simulate artificial skin (3M® surgical tape), and

FIG. 1a shows absorbance curves for the samples in a range between UVB (280-320 nm), UVA (320-400 nm) and up to 800 nm and 1B in a range between UVB (280-320 nm) and UVA (320-400 nm), and

FIG. 1C shows transmittance curves for the samples in a range between UVB (280-320 nm), UVA (320-400 nm) and up to 800 nm and 1D in a range between UVB (280-320 nm) and UVA (320-400 nm), wherein curve symbols: continuous line “bolt”—formulation 4 with scytonemin added; sample 1 - - - ; sample 2 -. -. -. and sample 3 - - -

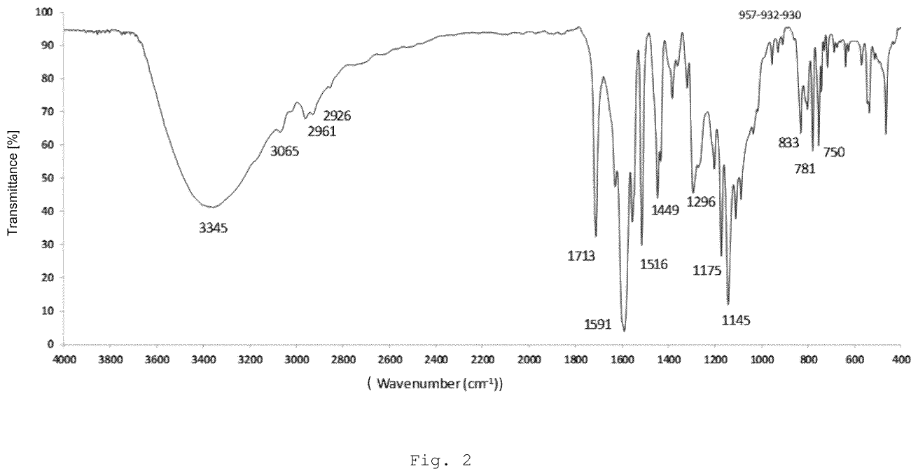

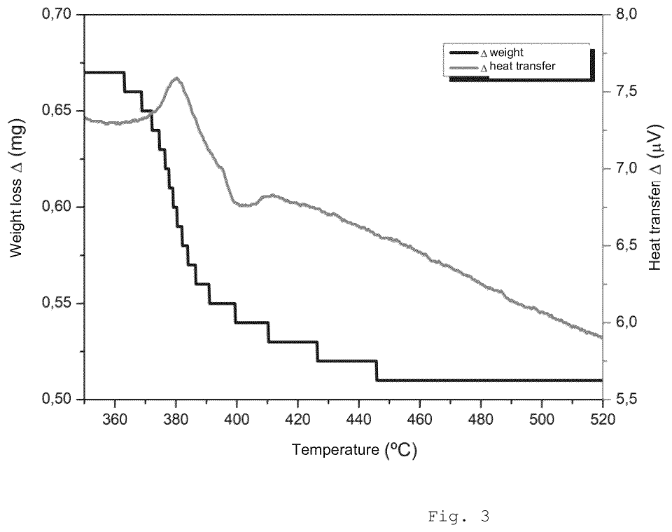

FIG. 2 illustrates the FTIR spectrum of the SCY sample,

FIG. 3 shows the weight loss curve depending on sample heating temperature (black curve) and the heat flow curve (gray curve) in a temperature range of 350-520° C. with maximum decomposition temperature at 380.3° C.,

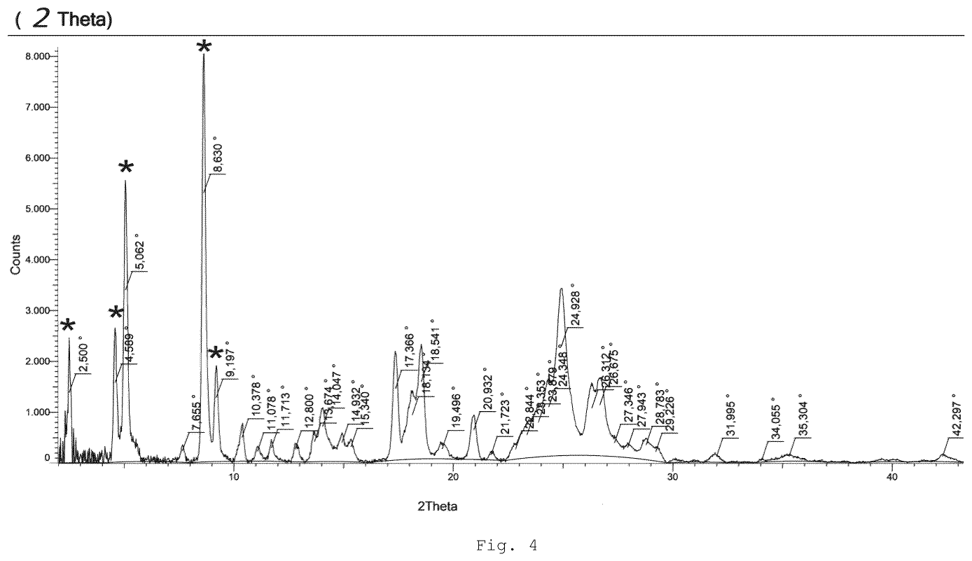

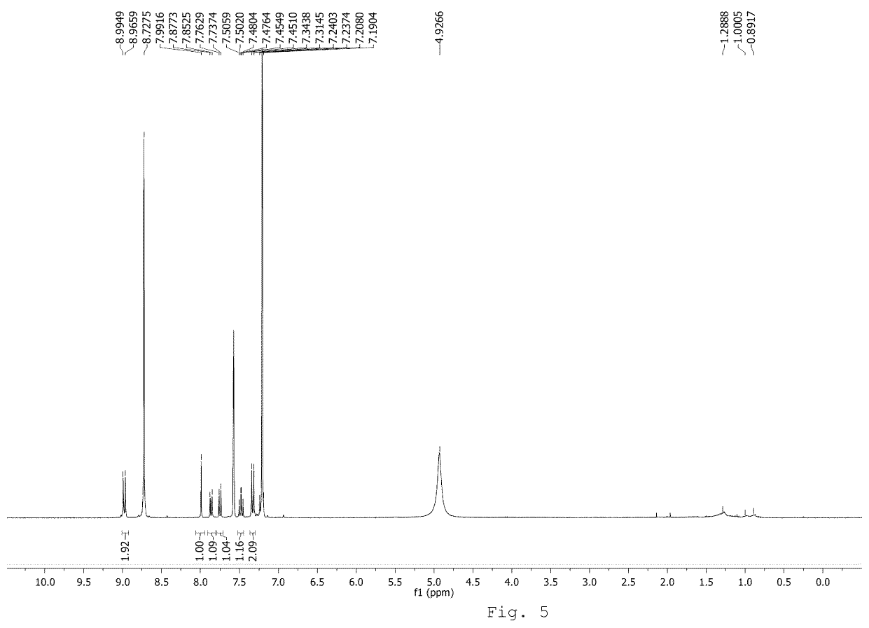

FIG. 4 shows an X-ray diffractogram obtained using PXRD (polycrystalline X-ray diffraction method) of the scytonemin form of the invention with the major peaks marked with “*”,

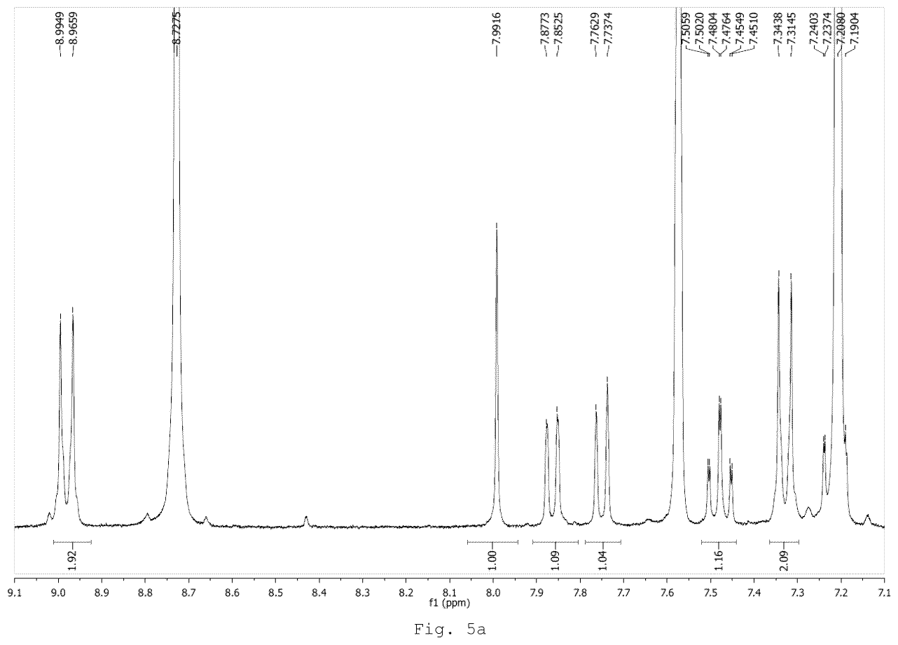

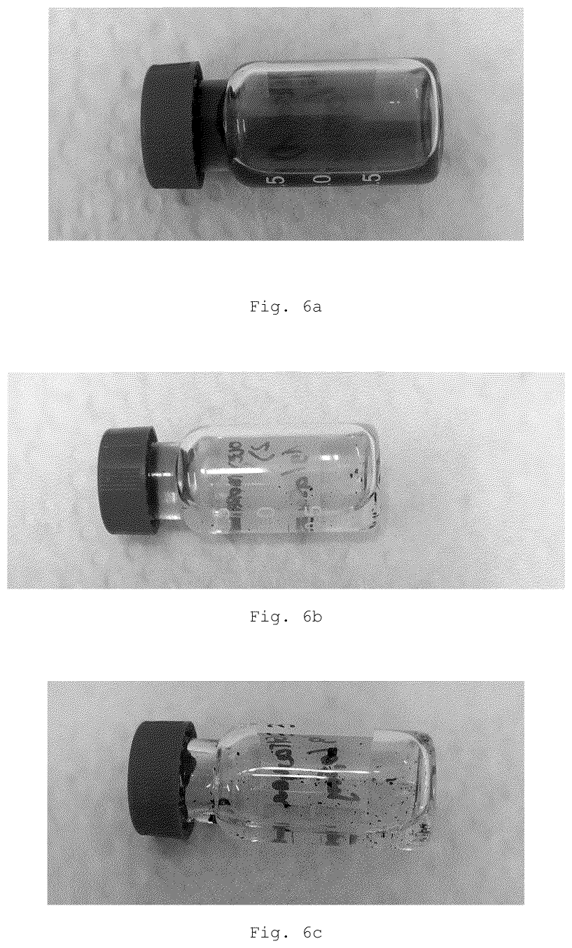

FIGS. 5 and 5a present a proton nuclear magnetic resonance (¹H NMR) spectrum of the scytonemin sample recorded in pyridine-d5, in the δ scale [ppm], wherein FIG. 5 contains a complete spectrum (range: −0.5 to 10.5 ppm), and FIG. 5a shows an extended range of 7.1-9.1 ppm, while

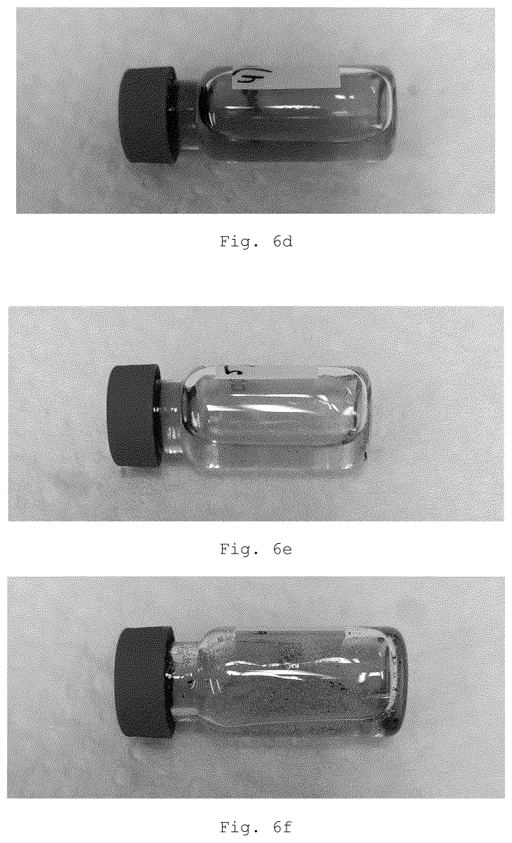

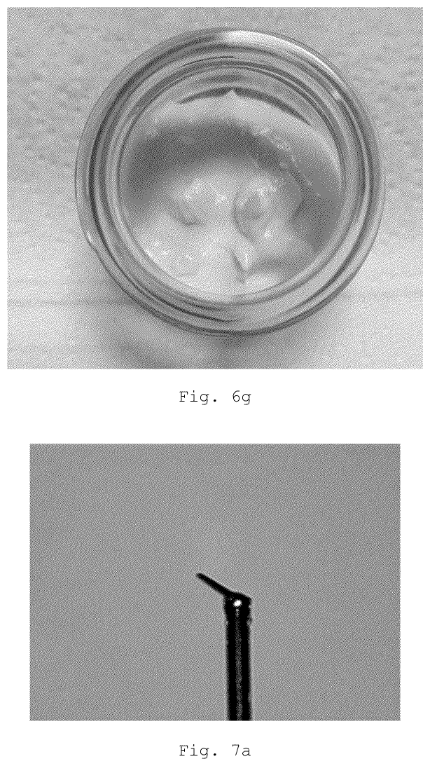

FIG. 6 presents results of investigation into the degree of scytonemin dispersion in selected solutions used in cosmetics disclosed in Example 2.2,

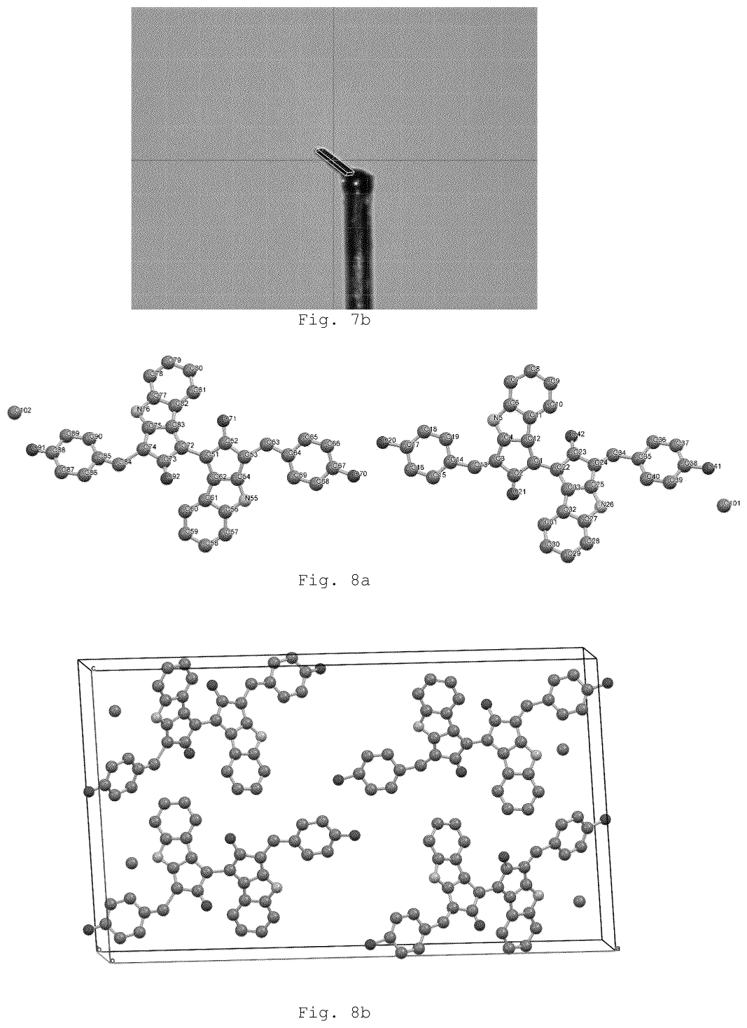

FIGS. 7a and 7b show the crystal described in Example 8,

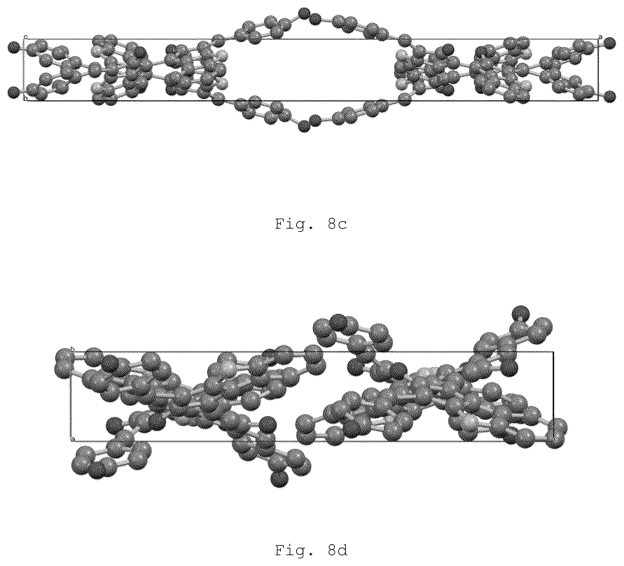

FIG. 8a-8d show graphic models of the SCY structure obtained using MERCURY software (Macrae et al., 2020): asymmetric unit (FIG. 8a), general view of unit cell packing (FIG. 8b), and unit cell packing, view along the direction (FIG. 8c) and along the direction (FIG. 8d)

### EXAMPLE 1

### 1.1 Preparation of Growth Medium

Preparation of the growth medium involved enrichment in micro- and macronutrients found in sandstone originating from the Nubian formation from which Cyanobacteria are sourced. Therefore, 200 g of sterilized stone was ground in an agate mortar and added per each 1000 mL of a pure BG11 medium according to Table 1. The resulting mixture was subsequently stirred for 24 hours at 25° C., subjected to final 5-hour sedimentation and filtered through a filter with a diameter of 25 mm and pore size of 0.2 μm (Cyclopore Track-Etch Membranes, Whatman). The resulting BG11 medium enriched in micro- and macronutrients from sandstone was heated to 60° C. Subsequently, dry agar in the final amount of 2% by weight was added to the resulting solution. Subsequently the entire contents were stirred until the agar dissolved and poured on Petri dishes (R), cooled to 25° C. and kept covered in sterile conditions for Cyanobacteria collection from the environment and seeding on plates with the medium.

1.1.1.

### Characteristics of the Stone

Stones with endolytic microorganisms originate from the Nubian Sandstone formation. X-day diffraction (XRD) was used for the quantitative analysis of the mineralogical composition of five samples of the stones with a total weight of 52 g. The sandstone had mean contents of: quartz 97.6%, muscovite-biotite 0.4%, apatite 1.2% and other minerals in trace quantities of 0.8%.

### 1.2 Collection of Cyanobacteria from the Environment

Two small stone fragments with endolytic colonization by two Cyanobacteria strains being the object of the present invention were scraped mechanically using a sterile lancet onto the Petri dishes of item 1.1 above so that two separate cultures were set up for two strains, enriched in micro- and macronutrients from Nubian sandstone. The Cyanobacteria were seeded at 25° C. in the light/dark regimen (12/12 hrs.) at 25° C., with light intensity in the PAR (photosynthetic active radiation, 400-700 nm) range of approx. 30-50 μmol photons m⁻² s⁻¹, provided by 18 W cool fluorescent lamps (PhilipsTLD18W/33). After 5-10 weeks, the agar dishes were tested for the presence of Cyanobacateria colonies using a Euromex Oxion Inverso OX.2053-PL light microscope+Cmex 3 camera.

### 1.3 Passaging

Gradual passaging of the biological material from stage 1.2 was performed on solid media obtained in stage 1.1 and was conducted from the additional agar content of initially 2% by weight of the medium to 0.5% finally, preferably in three intermediate stages with agar contents of 1.75%, 1.5%, 1%. The passaging time was 4 weeks for each intermediate and final stages at 25° C. with continuous PAR (400-700 nm) irradiation at 35 μmol photons m⁻² s⁻¹.

### 1.4 Culture

The resulting colonies from stage 1.3 for two strains from the medium with an agar content of 0.5% by weight were dissolved in an aqueous solution of the medium from stage 1.1 whose composition is disclosed in Table 1, which had not been modified using the addition of micro- and macronutrients from the stone and were incubated at 25° C. for 2 weeks with simultaneous continuous orbital shaking (20 rpm) using an IKA KS 501 Orbital Shaker and with continuous PAR irradiation (400-700 nm) at 35μ mol photons m⁻² s⁻¹ and two separate cultures for the two strains being the object of the invention were further maintained.

Preparation of a monoclonal stable culture of two strains of the invention in the medium of stage 1.1, not modified using the addition of micro- and macronutrients from the stone.

Cyanobacteria colonies isolated under the microscope were placed in an aqueous solution of the medium of stage 1.1 whose composition is listed in Table 1 without the addition of the stone at pH 8.2; temp. 25° C. and PAR light intensity of 20 μmol photons m⁻² s⁻¹ and were shaken at certain intervals for resuspension. Part (2 mL) of the culture was added every two weeks to 100 mL fresh standard medium of stage 1.1 without the addition of the stone to maintain a fresh culture. The photoperiod during cyanobacterial culture in the liquid medium was 10-12 hours of light and 12-14 hours of dark in a continuous or mixed mode.

### Induction of a Cyanobacteria Culture for Scytonemin Synthesis and Determination of its Productivity

The methodology was taken from Fleming and Castenholtz (2007). Briefly, Cyanobacteria solutions from the above medium, i.e. from stage 1.1, without the addition of the stone were filtered and the filters were placed on a BG-11 solid agar medium with an agar content of 2%. The dishes with the filters were subjected to PAR (65 μmol photons m⁻² s⁻¹, or 40 W/m²) and UV irradiation (1.8 W/m²). Some filters with irradiated Cyanobacteria were analyzed for scytonemin content every three days. Therefore, a spectrophotometry technique was used as presented below: absorption spectra of extracted (methanol/ethyl acetate (v/v 1:1)) scytonemin were obtained using an HP 8452A Diode Array single-beam spectrophotometer (Hewlett-Packard, Tokyo, Japan).

Absorbance values for the specific wavelength (maximum peaks for respective pigments) were selected for the semi-quantitative assay of scytonemin [mg/g dry weight (DW)] using trichromatic equations and extinction coefficients (Lichtenthaler, 1987).

### 1.5 Method for the Evaluation of Scytonemin Productivity of the Invention

The culture solutions containing Cyanobacteria from stage 1.4 after the end of culture were filtered using a 0.2 μm filter. The filters were placed on a BG-11 solid agar medium (2%). The dishes with the filters were subjected to PAR irradiation at 65 μmol photons m⁻² s⁻¹ (or 40 W/m²) and UVA irradiation at 1.8 W/m². Some filters with irradiated Cyanobacteria were analyzed for scytonemin content every three days. Therefore, a spectrophotometry technique was used as shown below:

Absorption spectra of extracted (methanol/ethyl acetate (v/v 1:1)) scytonemin (with other pigments) were obtained using an HP 8452A Diode Array single-beam spectrophotometer (Hewlett-Packard, Tokyo, Japan). Absorbance values for the specific wavelength (maximum peaks for respective pigments) were selected for the semi-quantitative assay of scytonemin [mg/g dry weight (DW)] using trichromatic equations and extinction coefficients (Lichtenthaler, 1987).

The resulting scytonemin productivity was at least 1.75% for the bacterial strain deposited in Banco Espanol de Algas Universidad de Las Palmas de GC under number BEA_IDA_0075B and for the bacterial strain deposited in Banco Espanol de Algas Universidad de Las Palmas de GC under number BEA_IDA_0068B as per dry weight of Cyanobacterium, that is, much higher than in the art in which it was between 0.03-0.09% scytonemin per dry weight of Cyanobacteria (DW) (Balskus 2011); et al., therefore, productivity was between 19 and 58 times as high.

### 1.6 Extraction and Purification of Scytonemin

The biomass obtained according to the description in the items above suspended in the culture liquid is separated by centrifugation (or filtration). The resulting biomass is subjected to preliminary purification in a chloroform:hexane mixture (v/v 1:1). In this stage, the biomass with the mixture of solvents is shaken for 10 minutes and sonicated, also for 10 minutes. This is subsequently centrifuged (6000 rpm for 10 min) and the supernatant is collected from above the sediment. Another fresh portion of the mixture of solvents is added to the sediment and the procedure is repeated. After another centrifugation, the supernatant from both centrifugations is merged and may be purified using a vacuum evaporator for reuse. The biomass after the first stage of purification is subsequently subjected to primary extraction in an ethyl acetate:methanol mixture (v/v 1:1) or in acetone. Centrifugation and sonication in 10-minute cycles is also used at this stage. Centrifugation follows every cycle and the supernatant is collected. Extraction is repeated with further fresh portions of the solvent until the supernatant starts to lose color (typically 3 to 5 times). The collected supernatant is subsequently evaporated using a vacuum evaporator) (40° C. for reuse. The dried residue after evaporation is subjected to the final purification procedure. The chloroform:hexane (v/v 1:1) is also used at this stage, with shaking, sonication and centrifugation. The number of purification stages depends on the degree of sample contamination and it is repeated until a clear colorless supernatant is obtained after centrifugation. When this effect is achieved, the sediment (scytonemin) is additionally washed with hexane twice. After the last centrifugation and collection of the supernatant from above the sediment, it is dried in a vacuum dryer (40° C.) and then weighed. The dried sediment is assayed by HPLC to assess the purity of the resulting product.

### EXAMPLE 2 USE OF SCYTONEMIN

### 2.1. Efficiency of Sun Protection

To demonstrate the efficiency of sun protection of various brands of sunscreens and the sunscreen product proposed by the applicant (sample 4) with scytonemin based on Cyanobacterium extracts as the active ingredient, a spectrophotometry technique was used. Therefore, commercially available sunscreen products and the tested sample 4 were analyzed for absorbance and transmittance in an experiment using simulation of human skin (3M surgical tape). The commercially available 3M® tape was applied on a 2×2 cm quartz glass tape onto which a thin layer of the products being evaluated was applied. The plates were tested for absorbance and transmittance after 20 minutes using a FLAME-S (Ocean Optics, Florida, USA) system and spectrometer.

The efficiency of sun protection of the scytonemin product of the invention is shown in FIG. 1 which presents comparative results of testing absorbance (A and B) and transmittance (C and D) for selected commercially available products with SPF (Sun Protection Factor) (Greiter, 1974) of 30 and 50 (samples 1-3) and sample 4 with scytonemin added. The tests were conducted using a thin-layer material that simulated artificial skin (3M® surgical tape). FIG. 1A shows absorbance curves for the samples in a range between UVB (280-320 nm), UVA (320-400 nm) and up to 800 nm and 1B in a range between UVB (280-320 nm) and UVA (320-400 nm), and FIG. 1C shows transmittance curves for the samples in a range between UVB (280-320 nm), UVA (320-400 nm) and up to 800 nm and 1D in a range between UVB (280-320 nm) and UVA (320-400 nm), wherein curve symbols: continuous line “bolt”—formulation 4 with scytonemin added; sample 1 - - - ; sample 2 -. -. -. and sample 3 - - - .

Commercially available products whose specific compositions are listed below were selected for comparative analysis:
Sample 1 2-Ethylhexyl 4-methoxycinnamate/Octinoxate+2-Hydroxy-4-methoxybenzophenone/Oxybenzone+titanium dioxide (TiO₂) [percentage contents in the product: 7.5%, 4% and 10%, respectively]+q.s. (quantum satis): glycerin+glycerol stearate+water+silica+alcohol.
Sample 2 titanium dioxide (TiO₂)+zinc oxide (ZnO) [percentage contents in the product: 10% and 17%, respectively]+q.s.: glycerin+glycerol stearate+water+silica+alcohol.
Sample 3 zinc oxide (ZnO)+(2-Ethylhexyl 4-methoxycinnamate)/Octinoxate [percentage contents in the product: 15.5% and 7.5%, respectively]+q.s.: glycerin+glycerol stearate+water+silica+alcohol.
Sample 4 0.8% SCYTONEMIN+Diprobase q.s. with the composition: white petrolatum, liquid paraffin, macrogol cetostearyl ether, cetostearyl alcohol, sodium dihydrogen phosphate dihydrate, chlorocresol, sodium hydroxide, concentrated phosphoric acid, purified water.

Observation: the formulation with scytonemin added showed high absorbance values and low transmittance values in the UVB and UVA range at a level similar to commercially available creams with SPF 30 and 50.

### 2.2 Testing the Degree of Scytonemin Dispersion in Selected Solutions Used in Cosmetics

2.2.1

0.5 mg scytonemin was weighed out on an analytical balance and suspended in 1 g of the solution:
1) Propylene glycol (INCI: Propylene Glycol)
2) Refined apricot oil (INCI: Prunus Armeniaca (Apricot) Kernel Oil)
3) Glycerin (INCI: Glycerin)
4) Isohexadecane (INCI: Isohexadecane)
5) 2-octyldodecan-1-ol ODD (INCI: Octyldodecanol)
6) SLP Emulsifier (INCI: Sorbitan Laurate/Polyglyceryl-4 Laurate/Dilauryl Citrate)

Subsequently, the sample was mixed using a shaker for approx. 1 min and maintained for 10 min in an ultrasonic bath to achieve a higher dispersion level.

### Results:

1. High dispersion level of the active ingredient; the glycol solution immediately turns brown-green; small particles of the suspension are seen (see FIG. 6a).
2. Very low dispersion level of the active ingredient in the solution; undissolved suspension is clearly seen which sediments over time (see FIG. 6b)
3. Low dispersion level of the active ingredient; suspension is clearly seen which gradually dissolves over time and slightly tints the solution (see FIG. 6c)
4. High dispersion level of the active ingredient. After suspending, an evenly dispersed grayish suspension is obtained; particles of the substance are seen (very fine) which sediment over time (see FIG. 6d)
5. Relatively high dispersion level of the active ingredient. A pale green solution was obtained; the suspension settling on the bottom is seen (see FIG. 6e)
6. Low dispersion level of the active ingredient in the emulsifier solution. Fine particles settling on the bottom are seen; the solution gradually became colored over time (see FIG. 6f).

### SUMMARY

The best dispersion level of the active ingredient was obtained in sample 1 at a concentration of 0.5 mg/g glycol. Decreasing dispersion levels were seen in successive samples in the following order (the samples are arranged from the highest to the lowest dispersion level of scytonemin in the matrix):

1>4>5>6>3>2.

2.2.2

### Preparation:

0.5 mg of scytonemin was weighed out on an analytical balance and suspended in 5 g of the composition:
7) glycerin+glycerol stearate+water+silica+alcohol

The sample was stirred for 5 min using a laboratory stirrer

### Result:

After mixing the raw material base with the active ingredient, a homogenous off-white base was obtained with visible scytonemin particles. The substance did not dissolve but disintegrated into smaller particles. The effect was similar as with sample 4 with isohexadecane (suspension)—see FIG. 6g.

1>4, 7>5>6>3>2.

### Example 3. Comparative Example—State of the Art—Standard Isolation and Culture Method

Using a standard method by Rippka et al. (1979) in which culture in the BG11 medium was used; Anahas and Muralitharan (2015) in which culture in the BG-11No medium was used; Singh et al. (2014) in which culture in the BG11 medium modified with 10 mM NaHCO₃ was used, bacteria of the strain being the object of the patent application could not be isolated, much less cultured. Bacterial colonies died early and biomass necessary to produce scytonemin could not be obtained.

### Example 4

### Fourier Transform Infrared Spectroscopy (FTIR) with Thermogravimetric/Differential Thermal Analysis TG/DTA)

SAMPLE IDENTIFICATION: SCY
Dark brown solid in a powder form
weight: 8.3 mg

### INTRODUCTION

A sample, hereinafter referred to as SCY, obtained from the strain being the object of the invention with deposit number BEA_IDA_0075B was prepared for further analysis using techniques described below: differential thermal analysis/thermogravimetry (TG/DTA) and Fourier transform infrared spectroscopy (FTIR) according to the specific procedures described below. No prior sample preparation was necessary for the analysis. Subsequently repeated experiments for a sample of strain BEA_IDA_0068B provided similar results. All results presented in the examples refer to the same substance obtained from two strains being the object of the invention.

A sample of SCY was stored until analysis in a closed container at room temperature.

### 4.1

### FTIR

209.9 mg KBr previously dried at 110° C. for five hours was weighed out for FTIR analysis and cooled in a vacuum desiccator to room temperature. 0.4 mg of the sample was added to KBr and the mixture was ground in an agate mortar under an infrared lamp to avoid absorption of moisture.

The spectrum was obtained in the following conditions:

### Equipment

Shimadzu FTIR 8400 spectrophotometer (Shimadzu, Kyoto, Japan). PIKE press (Pike Technologies, Madison, USA).

### Measurement Parameters:

Method: % Transmittance
Range: 400-4000 cm⁻¹
Apodization: Happ-Genzel
Scan number: 45
Resolution: 4.0

### Results:

The data were processed using Shimadzu software to obtain peak values. Peak values and bands were assigned according to the available literature (Pretsch, E. et al.: “Tablas para la elucidación estructural de compuestos orgánicos por métodos espectroscópicos” Ed. Alhambra. Madrid, 1980 and Flett, M. “Characteristic Frequencies of Chemical Groups in the Infra-red” Elsevier Monographs. Elsevier, 1963. FIG. 2 shows the FTIR spectrum of a sample of SCY (obtained in Example 1) with major bands marked. Table 2 shows wavenumber values assigned to the most probable identified functional groups/bonds.

TABLE 2

| | |
|---|---|
| 3345 | O—H stretching |
| 3065 | ═C—H stretching (alkenes, aromatics) |
| 1713 | C═O stretching |
| 1591 | C═C stretching (aromatics) |
| 1516 | C═C stretching (aromatics) |
| 1449 | C═C stretching (aromatics) |
| 1296 | O—H deformation |
| 1175 | C—O stretching |
| 957 | C—H in-plane deformation (aromatics) |
| 833 | C—H out-of-plane deformation (aromatics) |

Identical analysis was performed for the compound prepared in Example 1 from the strain deposited under number BEA_IDA_0068B. The resulting spectrum was identical as that in FIG. 2.

### 4.2 TG/DTA

3.7 mg of the sample was weighed out into an alumina crucible and 34.6 mg of pure gold wire (99.999%) was added to balance the rod weight with the microbalance counterweight.

### SAMPLE PREPARATION

No special preparation was needed for the analysis.

### APPARATUS

SETARAM SETSYS 6000 analyzer

### EXPERIMENTAL CONDITIONS

### Measurement Parameters

Argon flow at 2 atm
Sample weight: 3.7 mg
Crucible: 100 μL Al₂O₃
Reference crucible: Al₂O₃
Temperature ramp:

| Temperature (start- | | |
|---|---|---|
| end ) in [° C.] | Time [s] | Rate [° C./minute] |
| 25-25 | 300 | — |
| 25-900 | 5250 | 15 |
| 900-40 | 2580 | 20 |

### Results

Weight losses in successive stages were calculated based on the thermogravimetric curve, and the presence of exothermic and endothermic processes during sample heating were determined from the heat flow curve.

FIG. 3 shows the weight loss curve depending on sample heating temperature (black curve) and the heat flow curve (gray curve) in a temperature range of 350° C. to 520° C. A distinct weight loss of SCY (black curve) was seen in this temperature range. Thermal decomposition of a substance is an exothermic process (heat flow value increment on the gray curve), starts at 365.4° C. (vertical dashed line in FIG. 3) and achieves its maximum at 380.3° C. (vertical solid line in FIG. 3). It was found that a weight loss of 4.32% of SCY occurred in a temperature range of 365.4° C. to 380.3° C. related to an exothermic process, which confirmed decomposition temperature of SCY in this temperature range with a distinct maximum at 380.3° C. Weight loss of SCY was still seen above this temperature, associated with an endothermic process (decreasing values on the gray curve), which confirmed a process of gas release and restructuring of SCY decomposition products except for the temperature range of 405-412° C., in which an exothermic process was seen, associated with secondary decomposition of SCY decomposition products.

T range of SCY decomposition=365.4-380.3° C.

Maximum of weight loss=380.3° C.

Δweight=0.16 mg (4.32%)

Similar spectra were obtained for compounds obtained from the two strains being the object of the invention, cultured and isolated according to Example 1.

### EXAMPLE 5

### Polycrystalline X-Ray Diffraction (PXRD)

### Measurement Methodology

After extracting SCY from the two strains being the object of the invention, the solvent was evaporated and 8.3 mg of brown powder was obtained (crystalline form, form of the invention) with single needle-like crystals with a size of 2 to 20 micrometers or as their aggregates with radial arrangement of needle-like crystals. The characteristics of the crystalline material were observed using optical microscopy (AXIO Imager DM2 microscope, Zeiss, Carl Zeiss, Germany, Apochrome 63× lens, n=1.4 Zeiss).

PXRD powder analysis was performed in a crystalline material (form of the invention) composed of SCY, obtained from the two strains being the object of the invention, for which two similar PXRD diffractograms were recorded. The PXRD measurement was performed using a Bruker D8-Discover polycrystalline diffractometer. Powder diffractograms were obtained at room temp. with an X-ray tube as the X-ray source (Cu anode, Kα at 50 kV, 30 mA and collimator with a slit of 2 mm). Measurements were recorded in a continuous operating mode; 2 Theta angle scanning range between 2 and 60 degrees, measurement step of 0.02 degree, scanning rate of 0.7 sec/measurement step.

Diffrac.EVA v5.1 software was used for the analysis of the resulting diffractogram data.

The results of the analysis of powder X-ray diffraction spectra (form of the invention) presented in FIG. 4 are as follows:
1. The diffraction pattern for SCY does not show any amorphous phases.
2. The PXRD diffraction pattern contains approx. 35 significant diffraction peaks in the 2 Theta angle range of 2 to 43 degrees. Because the quality of the diffraction pattern was poorer and it was not possible to unambiguously determine (identify) peak parameters above this value, analysis was not performed.
3. Considering their characteristics (low full width at half maximum (FWHM), which confirms a high degree of crystallinity), at least five low-angle diffraction angles at the following 2 Theta angle values determined based on the available software: 2.500°, 4.589°, 5.062°, 8.630° and 9.197°, can be used to identify the material.

The peaks are marked in FIG. 4 with “*”.
4. The presence of other lower and broader peaks with higher FWHM values shows that a crystalline material with a lower degree of crystallinity (reduced crystallite size) occurs in the sample.
5. The recorded diffraction peaks are specific for SCY and may be used to identify the substance. Based on the available crystallographic databases of polycrystalline data, no other known substance with this diffraction pattern was found.

### EXAMPLE 6

### ¹H NMR (Proton Nuclear Resonance) Measurement of Scytonemin

The ¹H NMR spectrum of scytonemin (1.6 mg) was recorded in pyridine-d₅ (0.75 ml) on a Bruker Avance II 300 spectrometer at the basic frequency of 300.13 MHz at room temperature. δ chemical shifts are given in ppm, and values of J coupling constants in Hz. The spectrum was standardized with respect to the residual signal of H-2 protons of pyridine-d₅ at 8.727 ppm. The phase and baseline were corrected manually. Integration regions were selected in a similar fashion. Signals were assigned based on earlier literature data (Proteau et al., 1993). Experimental δ chemical shift data are consistent with the cited data. Due to rapid exchange of labile protons of —OH phenol groups, their signals were not included in the description. Signals from trace impurities (water and n-hexane) are found at δ 4.93 and about δ 1.0, respectively.

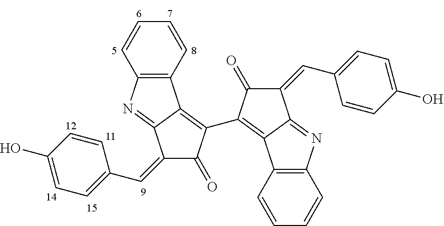 [SMILES: O=C1C(C2=C3C(=Nc4ccccc43)/C(=C\c3ccc(O)cc3)C2=O)=C2C(=Nc3ccccc32)/C1=C\c1ccc(O)cc1]

¹H NMR (pyridine-d₅) δ [ppm]: 8.98 (d, 2H, J 8.7; H-11, 15); 7.99 (s, 1H; H-9); 7.86 (d, 1H, J 7.5; H-8); 7.75 (d, 1H, J 7.6; H-5); 7.48 (td, 1H, J 7.6, 1.2; H-6); 7.33 (d, 2H, J 8.8; H-12, 14); 7.22 (m, 1H, H-7/overlaps with the residual signal of pyridine-d5 H-3 protons/).

FIGS. 5 and 5a show the proton spectrum (¹H NMR) of a scytonemin sample recorded in pyridine-d₅ in the δ scale [ppm], wherein FIG. 5 contains a complete spectrum (range of −0.5 to 10.5 ppm), and FIG. 5a contains an extended range of 7.1-9.1 ppm.

### EXAMPLE 7

The compound prepared in Example 1 was stored in room conditions (temp. 25° C.) for 10 months. Absorbance spectra before and after the storage test are identical, which confirms stability of the compound. In addition, the high stability of scytonemin was confirmed in papers (Fleming and Castenholz 2007) and (Rastogi and Incharoensakdi 2014) in which it was shown that scytonemin still had practically unchanged characteristic absorbance spectra after 2 months of continuous UVA irradiation (5 W/m2) or heating to 60° C. for 2 months. The crystalline form of scytonemin of the invention is stable.

### EXAMPLE 8

### Determination of the Scytonemin Structure Model (SCY) Using X-Ray Diffraction (XRD)

### Preparation of a Monocrystalline Sample of SCY

A monocrystalline sample of scytonemin for analysis using X-ray diffraction (XRD) was prepared by crystallization in the tetrahydrofuran (THF)-ethanol (EtOH) system in a 2:1 volumetric ratio. Approx. 30 mg of the compound and 12 mL of the THF-EtOH mixture was used in the process. The sample was initially dissolved in 8 mL THF, and subsequently, after 4 mL EtOH was added, the resulting solution was slowly (approx. 7 days) concentrated by free evaporation at room temperature.

### Data Collection and Reduction

One dark brown parallelepiped crystal with dimensions of 0.011×0.035×0.131 mm was selected from the test sample (FIGS. 7a and 7b).

Diffraction data for the selected SCY crystal were collected at 100 K using a Rigaku Oxford Diffraction Synergy-S four-cycle diffractometer equipped with a CuKα radiation source (1.54184 Å), graphite monochromator and an Oxford CryoStream 800 sample cooling system for low-temperature measurements. Refinement of cell parameters and data reduction were performed using software from the diffractometer manufacturer (Rigaku Oxford Diffraction, 2018).

### Structure Solving and Refining

The phase problem was solved by intrinsic phasing and atom positions in the structure model were determined using SHELXT (Sheldrick, 2015—Section A). Considering the quality of diffraction data, full-matrix refinement positions and isotropic atomic displacement parameters of non-hydrogen atoms based on structure factor squares (F² (hkl)) was only performed. To improve structure refinement and correct molecular geometry parameters, geometric constraints for benzene rings (AFIX 66) and terminal five-members rings having carbonyl groups (AFIX 56) were used.

Structure model refinement and additional calculations were performed using SHELXL2014 (Sheldrick, 2015-Section C) Parameters of the diffraction measurement, crystal lattice and structure model refinement for SCY are listed in Table 3. Parameters of the geometrically determined structure model of SCY are listed in Tables 4-7. These are, respectively: atomic coordinates expressed as fractions of unit cell parameters (×10⁴) and equivalent isotropic atomic displacement parameters U_{eq} (Å²×10³) for SCY, wherein V_{eq} values are defined as 1/3 of the trace of the orthogonalized U_{IJ} tensor (Table 4), bond lengths (Table 5) as well as valence (Table 6) and torsion angles (Table 7).

Graphic representations of the SCY structure model (FIGS. 8a-d) were obtained using MERCURY software (Macrae et al., 2020): asymmetric unit (FIG. 8a),

general view of unit cell packing (FIG. 8b) and unit cell packing, view along the direction (FIG. 8c) and along the direction (FIG. 8d).

TABLE 3

| | |
|---|---|
| Name | SCY |
| Molecular formula | C₃₆H₂₀N₂O₄ |
| Molecular weight/g mol⁻¹ | 544.57 |
| Measurement temperature/K | 100.00(15) |
| Crystal system | monoclinic |
| Space group | Pc |
| a/Å | 35.5024(13) |
| b/Å | 3.78158(10) |
| c/Å | 20.4422(6) |
| α/° | 90 |
| β/° | 95.338(3) |
| γ/° | 90 |
| Unit cell volume/Å³ | 2732.57(15) |
| Z | 4 |
| Density_{calc}/g cm⁻³ | 1.304 |
| μ/mm⁻¹ | 0.716 |
| Crystal dimensions/mm³ | 0.131 × 0.035 × 0.011 |
| Radiation source | Cukα (λ = 1.54184) |
| 2Θ angle range for recorded | 5 to 149.748. |
| data/° | |
| Ranges of determined hkl | −41 ≤ h ≤ 44, −4 ≤ k ≤ 4, −24 ≤ |
| parameters | l ≤ 24 |
| Number of reflections recorded | 6816 |
| Number of symmetrically | 6816 [R_{sigma} = 0.0239] |
| independent reflections | |
| recorded | |
| Number of reflections/number | 6816/16/215 |
| of constraints/number of | |
| refined parameters | |
| Structure model goodness of | 3.675 |
| fit parameter based on F(hkl)² | |
| R factors [I ≥ 2σ (I)] | R₁ = 0.2424, wR₂ = 0.5534 |
| Highest/lowest value in the | 2.38/−1.65 |
| difference electron density | |
| map/e Å⁻³ | |

TABLE 4

| Atom | x | | y | | z | | U_{eq} | |
|---|---|---|---|---|---|---|---|---|
| C1 | 7477 | (4) | −3830 | (50) | 6963 | (8) | 30 | (4) |
| C2 | 7274 | (5) | −2300 | (50) | 6402 | (7) | 43 | (6) |
| C3 | 6889 | (4) | −2040 | (60) | 6526 | (10) | 37 | (5) |
| C4 | 6853 | (4) | −3410 | (70) | 7164 | (10) | 68 | (9) |
| C12 | 7217 | (5) | −4520 | (60) | 7434 | (7) | 39 | (5) |
| N5 | 6580 | (6) | −2680 | (60) | 7543 | (9) | 39 | (5) |
| C11 | 7088 | (5) | −5090 | (60) | 8061 | (6) | 55 | (8) |
| C6 | 6716 | (5) | −4110 | (70) | 8129 | (8) | 47 | (7) |
| C7 | 6568 | (4) | −4510 | (70) | 8731 | (10) | 62 | (8) |
| C8 | 6792 | (5) | −5880 | (70) | 9265 | (7) | 56 | (8) |
| C9 | 7164 | (5) | −6860 | (50) | 9197 | (6) | 37 | (5) |
| C10 | 7312 | (4) | −6460 | (50) | 8595 | (7) | 21 | (4) |
| C13 | 6634 | (14) | −210 | (150) | 6110 | (30) | 80 | (12) |
| C14 | 6238 | (6) | 790 | (70) | 5931 | (12) | 41 | (6) |
| C15 | 6071 | (10) | 1590 | (120) | 5306 | (12) | 95 | (16) |
| C16 | 5694 | (11) | 2630 | (150) | 5220 | (20) | 110 | (20) |
| C17 | 5484 | (8) | 2870 | (170) | 5760 | (30) | 140 | (30) |
| C18 | 5650 | (10) | 2070 | (170) | 6380 | (20) | 180 | (50) |
| C19 | 6027 | (10) | 1030 | (110) | 6470 | (13) | 72 | (10) |
| O20 | 5077 | (9) | 3230 | (90) | 5546 | (18) | 72 | (8) |
| O21 | 7389 | (10) | −1730 | (100) | 5910 | (20) | 83 | (9) |
| C22 | 7872 | (4) | −4610 | (60) | 7096 | (9) | 43 | (6) |
| C23 | 8059 | (5) | −4180 | (60) | 7736 | (7) | 38 | (6) |
| C24 | 8437 | (5) | −5380 | (50) | 7725 | (8) | 32 | (5) |
| C25 | 8484 | (4) | −6570 | (60) | 7079 | (9) | 40 | (6) |
| C33 | 8135 | (6) | −6090 | (60) | 6690 | (6) | 49 | (7) |
| N26 | 8740 | (8) | −7660 | (120) | 6664 | (12) | 79 | (10) |
| C32 | 8180 | (4) | −7000 | (50) | 6027 | (6) | 29 | (5) |
| C27 | 8550 | (4) | −8240 | (50) | 6046 | (7) | 34 | (5) |
| C28 | 8693 | (4) | −9390 | (50) | 5473 | (9) | 36 | (5) |
| C29 | 8467 | (6) | −9310 | (60) | 4881 | (7) | 48 | (7) |
| C30 | 8097 | (5) | −8070 | (70) | 4861 | (6) | 65 | (10) |
| C31 | 7954 | (4) | −6920 | (60) | 5434 | (8) | 48 | (7) |
| C34 | 8684 | (5) | −5060 | (50) | 8250 | (9) | 17 | (3) |
| C35 | 9093 | (5) | −6120 | (70) | 8481 | (9) | 51 | (7) |
| C36 | 9236 | (4) | −5740 | (50) | 9133 | (8) | 34 | (5) |
| C37 | 9598 | (4) | −6940 | (50) | 9338 | (8) | 25 | (4) |
| C38 | 9817 | (4) | −8530 | (60) | 8890 | (10) | 82 | (12) |
| C39 | 9673 | (6) | −8910 | (70) | 8238 | (10) | 44 | (6) |
| C40 | 9311 | (7) | −7710 | (80) | 8033 | (8) | 130 | (30) |
| O41 | 10201 | (6) | −9340 | (60) | 9131 | (12) | 49 | (5) |
| O42 | 7964 | (6) | −2120 | (50) | 8222 | (10) | 41 | (4) |
| C51 | 2566 | (3) | 13380 | (50) | 2948 | (7) | 44 | (6) |
| C52 | 2750 | (4) | 11940 | (40) | 3534 | (5) | 26 | (4) |
| C53 | 3136 | (3) | 11400 | (40) | 3433 | (6) | 17 | (3) |
| C54 | 3190 | (3) | 12500 | (40) | 2785 | (6) | 16 | (3) |
| C62 | 2838 | (4) | 13730 | (50) | 2485 | (5) | 30 | (4) |
| N55 | 3453 | (6) | 12750 | (70) | 2335 | (10) | 47 | (6) |
| C61 | 2864 | (4) | 14900 | (50) | 1841 | (6) | 26 | (4) |
| C56 | 3247 | (4) | 14290 | (60) | 1805 | (7) | 34 | (5) |
| C57 | 3412 | (4) | 15100 | (70) | 1232 | (10) | 68 | (10) |
| C58 | 3193 | (6) | 16510 | (70) | 696 | (8) | 51 | (7) |
| C59 | 2810 | (6) | 17130 | (70) | 733 | (8) | 71 | (10) |
| C60 | 2645 | (4) | 16320 | (70) | 1305 | (9) | 58 | (8) |
| C63 | 3389 | (5) | 10270 | (50) | 4038 | (9) | 19 | (3) |
| C64 | 3783 | (3) | 9350 | (60) | 4039 | (8) | 81 | (12) |
| C65 | 3921 | (4) | 8090 | (50) | 4654 | (7) | 36 | (5) |
| C66 | 4301 | (4) | 7240 | (40) | 4779 | (6) | 21 | (4) |
| C67 | 4545 | (3) | 7660 | (50) | 4290 | (8) | 38 | (5) |
| C68 | 4407 | (4) | 8920 | (50) | 3675 | (7) | 36 | (5) |
| C69 | 4026 | (4) | 9770 | (50) | 3549 | (7) | 29 | (4) |
| O70 | 4886 | (11) | 5840 | (110) | 4330 | (20) | 89 | (10) |
| O71 | 2577 | (4) | 11830 | (40) | 4085 | (7) | 18 | (3) |
| C72 | 2128 | (3) | 14310 | (50) | 2856 | (7) | 41 | (5) |
| C73 | 1915 | (4) | 13840 | (40) | 2239 | (6) | 20 | (4) |
| C74 | 1539 | (4) | 14990 | (60) | 2303 | (8) | 58 | (8) |
| C75 | 1520 | (4) | 16160 | (50) | 2959 | (9) | 37 | (5) |
| C83 | 1884 | (4) | 15740 | (50) | 3301 | (6) | 23 | (4) |
| N76 | 1276 | (4) | 17990 | (40) | 3265 | (7) | 21 | (3) |
| C82 | 1841 | (4) | 17100 | (40) | 3931 | (5) | 35 | (5) |
| C77 | 1470 | (4) | 18330 | (50) | 3868 | (5) | 26 | (4) |
| C78 | 1309 | (3) | 19640 | (50) | 4413 | (8) | 49 | (7) |
| C79 | 1519 | (4) | 19700 | (40) | 5023 | (6) | 23 | (4) |
| C80 | 1889 | (4) | 18470 | (40) | 5086 | (5) | 20 | (4) |
| C81 | 2050 | (3) | 17170 | (40) | 4540 | (6) | 21 | (4) |
| C84 | 1242 | (6) | 15190 | (60) | 1567 | (12) | 29 | (4) |
| C85 | 896 | (4) | 16160 | (50) | 1462 | (8) | 38 | (6) |
| C86 | 755 | (5) | 15490 | (60) | 817 | (8) | 64 | (9) |
| C87 | 390 | (5) | 16520 | (60) | 595 | (7) | 49 | (7) |
| C88 | 165 | (4) | 18230 | (50) | 1019 | (9) | 27 | (4) |
| C89 | 305 | (4) | 18890 | (50) | 1665 | (8) | 63 | (9) |
| C90 | 671 | (4) | 17860 | (50) | 1886 | (7) | 21 | (4) |
| O91 | −169 | (7) | 18650 | (70) | 817 | (13) | 53 | (5) |
| O92 | 2030 | (5) | 12180 | (50) | 1792 | (9) | 33 | (4) |
| C101 | 10609 | (7) | −12320 | (60) | 8307 | (12) | 29 | (5) |
| C102 | −592 | (11) | 22390 | (110) | 1630 | (20) | 59 | (8) |

TABLE 5

| Atom | Atom | Length/Å | Atom | Atom | Length/Å |
|---|---|---|---|---|---|
| C1 | C2 | 1.4200 | C51 | C52 | 1.4200 |
| C1 | C12 | 1.4200 | C51 | C62 | 1.4200 |
| C1 | C22 | 1.434 (18) | C51 | C72 | 1.586 (15) |
| C2 | C3 | 1.4200 | C52 | C53 | 1.4200 |
| C2 | 021 | 1.13 (4) | C52 | 071 | 1.333 (17) |
| C3 | C4 | 1.4200 | C53 | C54 | 1.4200 |
| C3 | C13 | 1.36 (6) | C53 | C63 | 1.52 (2) |
| C4 | C12 | 1.4200 | C54 | C62 | 1.4200 |
| C4 | N5 | 1.325 (18) | C54 | N55 | 1.373 (18) |
| C12 | C11 | 1.418 (15) | C62 | C61 | 1.400 (14) |
| N5 | C6 | 1.362 (18) | N55 | C56 | 1.380 (19) |
| C11 | C6 | 1.3900 | C61 | C56 | 1.3900 |
| C11 | C10 | 1.3900 | C61 | C60 | 1.3900 |
| C6 | C7 | 1.3900 | C56 | C57 | 1.3900 |
| C7 | C8 | 1.3900 | C57 | C58 | 1.3900 |
| C8 | C9 | 1.3900 | C58 | C59 | 1.3900 |
| C9 | C10 | 1.3900 | C59 | C60 | 1.3900 |
| C13 | C14 | 1.47 (5) | C63 | C64 | 1.44 (2) |
| C14 | C15 | 1.3900 | C64 | C65 | 1.3900 |
| C14 | C19 | 1.3900 | C64 | C69 | 1.3900 |
| C15 | C16 | 1.3900 | C65 | C66 | 1.3900 |
| C16 | C17 | 1.3900 | C66 | C67 | 1.3900 |
| C17 | C18 | 1.3900 | C67 | C68 | 1.3900 |
| C17 | 020 | 1.48 (4) | C67 | 070 | 1.39 (4) |
| C18 | C19 | 1.3900 | C68 | C69 | 1.3900 |
| C22 | C23 | 1.4200 | C72 | C73 | 1.4200 |
| C22 | C33 | 1.4200 | C72 | C83 | 1.4200 |
| C23 | C24 | 1.4200 | C73 | C74 | 1.4200 |
| C23 | 042 | 1.33 (2) | C73 | 092 | 1.21 (2) |
| C24 | C25 | 1.4200 | C74 | C75 | 1.4200 |
| C24 | C34 | 1.33 (2) | C74 | C84 | 1.76 (3) |
| C25 | C33 | 1.4200 | C75 | C83 | 1.4200 |
| C25 | N26 | 1.36 (2) | C75 | N76 | 1.314 (15) |
| C33 | C32 | 1.422 (15) | C83 | C82 | 1.409 (13) |
| N26 | C27 | 1.39 (2) | N76 | C77 | 1.360 (15) |
| C32 | C27 | 1.3900 | C82 | C77 | 1.3900 |
| C32 | C31 | 1.3900 | C82 | C81 | 1.3900 |
| C27 | C28 | 1.3900 | C77 | C78 | 1.3900 |
| C28 | C29 | 1.3900 | C78 | C79 | 1.3900 |
| C29 | C30 | 1.3900 | C79 | C80 | 1.3900 |
| C30 | C31 | 1.3900 | C80 | C81 | 1.3900 |
| C34 | C35 | 1.54 (2) | C84 | C85 | 1.28 (2) |
| C35 | C36 | 1.3900 | C85 | C86 | 1.3900 |
| C35 | C40 | 1.3900 | C85 | C90 | 1.3900 |
| C36 | C37 | 1.3900 | C86 | C87 | 1.3900 |
| C37 | C38 | 1.3900 | C87 | C88 | 1.3900 |
| C38 | C39 | 1.3900 | C88 | C89 | 1.3900 |
| C38 | 041 | 1.44 (3) | C88 | 091 | 1.23 (3) |
| C39 | C40 | 1.3900 | C89 | C90 | 1.3900 |

TABLE 6

| Atom | Atom | Atom | Angle/° | Atom | Atom | Atom | Angle/ ° |
|---|---|---|---|---|---|---|---|
| C2 | C1 | C22 | 130.7 (14) | C52 | C51 | C62 | 108.0 |
| C12 | C1 | C2 | 108.0 | C52 | C51 | C72 | 123.7 (12) |
| C12 | C1 | C22 | 121.3 (14) | C62 | C51 | C72 | 128.3 (11) |
| C1 | C2 | C3 | 108.0 | C53 | C52 | C51 | 108.0 |
| O21 | C2 | C1 | 126 (2) | 071 | C52 | C51 | 120.8 (12) |
| O21 | C2 | C3 | 125 (2) | 071 | C52 | C53 | 130.2 (11) |
| C2 | C3 | C13 | 121 (3) | C52 | C53 | C54 | 108.0 |
| C4 | C3 | C2 | 108.0 | C52 | C53 | C63 | 115.3 (11) |
| C4 | C3 | C13 | 130 (3) | C54 | C53 | C63 | 136.1 (11) |
| C12 | C4 | C3 | 108.0 | C62 | C54 | C53 | 108.0 |
| N5 | C4 | C3 | 126.4 (17) | N55 | C54 | C53 | 143.5 (12) |
| N5 | C4 | C12 | 121.5 (17) | N55 | C54 | C62 | 108.4 (12) |
| C1 | C12 | C4 | 108.0 | C54 | C62 | C51 | 108.0 |
| C11 | C12 | C1 | 157.9 (15) | C61 | C62 | C51 | 139.9 (12) |
| C11 | C12 | C4 | 92.2 (14) | C61 | C62 | C54 | 112.1 (12) |
| C4 | N5 | C6 | 102.3 (17) | C54 | N55 | C56 | 102.1 (15) |
| C6 | C11 | C12 | 116.0 (14) | C56 | C61 | C62 | 98.5 (12) |
| C6 | C11 | C10 | 120.0 | C56 | C61 | C60 | 120.0 |
| C10 | C11 | C12 | 124.0 (14) | C60 | C61 | C62 | 141.5 (12) |
| N5 | C6 | C11 | 106.3 (14) | N55 | C56 | C61 | 118.6 (14) |
| N5 | C6 | C7 | 133.5 (14) | N55 | C56 | C57 | 121.3 (14) |
| C11 | C6 | C7 | 120.0 | C57 | C56 | C61 | 120.0 |
| C8 | C7 | C6 | 120.0 | C56 | C57 | C58 | 120.0 |
| C9 | C8 | C7 | 120.0 | C57 | C58 | C59 | 120.0 |
| C8 | C9 | C10 | 120.0 | C58 | C59 | C60 | 120.0 |
| C9 | C10 | C11 | 120.0 | C59 | C60 | C61 | 120.0 |
| C14 | C13 | C3 | 148 (5) | C64 | C63 | C53 | 124.7 (15) |
| C15 | C14 | C13 | 127 (3) | C65 | C64 | C63 | 110.1 (12) |
| C15 | C14 | C19 | 120.0 | C65 | C64 | C69 | 120.0 |
| C19 | C14 | C13 | 113 (3) | C69 | C64 | C63 | 129.8 (12) |
| C16 | C15 | C14 | 120.0 | C66 | C65 | C64 | 120.0 |
| C15 | C16 | C17 | 120.0 | C65 | C66 | C67 | 120.0 |
| C16 | C17 | C18 | 120.0 | C68 | C67 | C66 | 120.0 |
| C16 | C17 | 020 | 111 (4) | 070 | C67 | C66 | 120 (2) |
| C18 | C17 | 020 | 128 (4) | 070 | C67 | C68 | 117 (2) |
| C19 | C18 | C17 | 120.0 | C67 | C68 | C69 | 120.0 |
| C18 | C19 | C14 | 120.0 | C68 | C69 | C64 | 120.0 |
| C23 | C22 | C1 | 121.1 (14) | C73 | C72 | C51 | 120.8 (11) |
| C23 | C22 | C33 | 108.0 | C83 | C72 | C51 | 131.1 (12) |
| C33 | C22 | C1 | 130.8 (14) | C83 | C72 | C73 | 108.0 |
| C22 | C23 | C24 | 108.0 | C74 | C73 | C72 | 108.0 |
| 042 | C23 | C22 | 128.9 (16) | 092 | C73 | C72 | 123.1 (14) |
| 042 | C23 | C24 | 120.3 (16) | 092 | C73 | C74 | 127.7 (14) |
| C25 | C24 | C23 | 108.0 | C73 | C74 | C75 | 108.0 |
| C34 | C24 | C23 | 120.8 (15) | C73 | C74 | C84 | 115.5 (12) |
| C34 | C24 | C25 | 131.0 (15) | C75 | C74 | C84 | 135.8 (12) |
| C24 | C25 | C33 | 108.0 | C83 | C75 | C74 | 108.0 |
| N26 | C25 | C24 | 144.7 (17) | N76 | C75 | C74 | 135.0 (13) |
| N26 | C25 | C33 | 106.8 (17) | N76 | C75 | C83 | 115.8 (13) |
| C22 | C33 | C32 | 142.3 (15) | C75 | C83 | C72 | 108.0 |
| C25 | C33 | C22 | 108.0 | C82 | C83 | C72 | 148.2 (12) |
| C25 | C33 | C32 | 109.4 (15) | C82 | C83 | C75 | 103.7 (12) |
| C25 | N26 | C27 | 108 (2) | C75 | N76 | C77 | 100.4 (13) |
| C27 | C32 | C33 | 104.1 (14) | C77 | C82 | C83 | 102.8 (11) |
| C27 | C32 | C31 | 120.0 | C77 | C82 | C81 | 120.0 |
| C31 | C32 | C33 | 135.9 (14) | C81 | C82 | C83 | 136.9 (11) |
| C32 | C27 | N26 | 110.5 (15) | N76 | C77 | C82 | 116.6 (12) |
| C28 | C27 | N26 | 128.9 (15) | N76 | C77 | C78 | 123.3 (12) |
| C28 | C27 | C32 | 120.0 | C82 | C77 | C78 | 120.0 |
| C27 | C28 | C29 | 120.0 | C77 | C78 | C79 | 120.0 |
| C30 | C29 | C28 | 120.0 | C80 | C79 | C78 | 120.0 |
| C29 | C30 | C31 | 120.0 | C81 | C80 | C79 | 120.0 |
| C30 | C31 | C32 | 120.0 | C80 | C81 | C82 | 120.0 |
| C24 | C34 | C35 | 139.2 (18) | C85 | C84 | C74 | 130.3 (19) |
| C36 | C35 | C34 | 121.2 (14) | C84 | C85 | C86 | 111.1 (15) |
| C36 | C35 | C40 | 120.0 | C84 | C85 | C90 | 128.8 (15) |
| C40 | C35 | C34 | 118.7 (14) | C86 | C85 | C90 | 120.0 |
| C35 | C36 | C37 | 120.0 | C85 | C86 | C87 | 120.0 |
| C38 | C37 | C36 | 120.0 | C86 | C87 | C88 | 120.0 |
| C37 | C38 | 041 | 115.8 (15) | C89 | C88 | C87 | 120.0 |
| C39 | C38 | C37 | 120.0 | 091 | C88 | C87 | 116.3 (18) |
| C39 | C38 | 041 | 123.8 (15) | 091 | C88 | C89 | 123.1 (18) |
| C38 | C39 | C40 | 120.0 | C90 | C89 | C88 | 120.0 |
| C39 | C40 | C35 | 120.0 | C89 | C90 | C85 | 120.0 |

TABLE 7

| A | B | C | D | Angle/° | A | B | C | D | Angle/° |
|---|---|---|---|---|---|---|---|---|---|
| C1 | C2 | C3 | C4 | 0.0 | C51 | C52 | C53 | C54 | 0.0 |
| C1 | C2 | C3 | C13 | 171 (3) | C51 | C52 | C53 | C63 | 172.9 (16) |
| C1 | C12 | C11 | C6 | 152 (4) | C51 | C62 | C61 | C56 | 179.0 (17) |
| C1 | C12 | C11 | C10 | −27 (6) | C51 | C62 | C61 | C60 | −1 (3) |
| C1 | C22 | C23 | C24 | 176 (2) | C51 | C72 | C73 | C74 | −178 (2) |
| C1 | C22 | C23 | 042 | −23 (3) | C51 | C72 | C73 | 092 | 14 (2) |
| C1 | C22 | C33 | C25 | −175 (3) | C51 | C72 | C83 | C75 | 178 (2) |
| C1 | C22 | C33 | C32 | 11 (4) | C51 | C72 | C83 | C82 | 2 (3) |
| C2 | C1 | C12 | C4 | 0.0 | C52 | C51 | C62 | C54 | 0.0 |
| C2 | C1 | C12 | C11 | −155 (5) | C52 | C51 | C62 | C61 | −180 (2) |
| C2 | C1 | C22 | C23 | 142.9 (16) | C52 | C51 | C72 | C73 | −144.8 (14) |
| C2 | C1 | C22 | C33 | −42 (3) | C52 | C51 | C72 | C83 | 38 (2) |
| C2 | C3 | C4 | C12 | 0.0 | C52 | C53 | C54 | C62 | 0.0 |
| C2 | C3 | C4 | N5 | 157 (3) | C52 | C53 | C54 | N55 | −178 (3) |
| C2 | C3 | C13 | C14 | 173 (7) | C52 | C53 | C63 | C64 | 175.7 (18) |
| C3 | C4 | C12 | C1 | 0.0 | C53 | C54 | C62 | C51 | 0.0 |
| C3 | C4 | C12 | C11 | 171 (2) | C53 | C54 | C62 | C61 | 179.7 (16) |
| C3 | C4 | N5 | C6 | −169.2 (19) | C53 | C54 | N55 | C56 | 179.1 (18) |
| C3 | C13 | C14 | C15 | −150 (7) | C53 | C63 | C64 | C65 | 173.7 (14) |
| C3 | C13 | C14 | C19 | 32 (10) | C53 | C63 | C64 | C69 | 11 (3) |
| C4 | C3 | C13 | C14 | −19 (11) | C54 | C53 | C63 | C64 | −14 (3) |
| C4 | C12 | C11 | C6 | −5.0 (15) | C54 | C62 | C61 | C56 | −0.5 (16) |
| C4 | C12 | C11 | C10 | 176.5 (16) | C54 | C62 | C61 | C60 | 179.8 (17) |
| C4 | N5 | C6 | C11 | 9 (2) | C54 | N55 | C56 | C61 | −4 (3) |
| C4 | N5 | C6 | C7 | −175 (2) | C54 | N55 | C56 | C57 | 178.7 (14) |
| C12 | C1 | C2 | C3 | 0.0 | C62 | C51 | C52 | C53 | 0.0 |
| C12 | C1 | C2 | 021 | −172 (3) | C62 | C51 | C52 | 071 | 169.7 (16) |
| C12 | C1 | C22 | C23 | −36 (3) | C62 | C51 | C72 | C73 | 34 (2) |
| C12 | C1 | C22 | C33 | 139.2 (18) | C62 | C51 | C72 | C83 | 143.8 (14) |
| C12 | C4 | N5 | C6 | −15 (3) | C62 | C54 | N55 | C56 | 3 (2) |
| C12 | C11 | C6 | N5 | −2 (2) | C62 | C61 | C56 | N55 | 3 (2) |
| C12 | C11 | C6 | C7 | −179 (2) | C62 | C61 | C56 | C57 | −179.8 (15) |
| C12 | C11 | C10 | C9 | 178 (2) | C62 | C61 | C60 | C59 | 180 (2) |
| N5 | C4 | C12 | C1 | −158 (3) | N55 | C54 | C62 | C51 | 178.5 (18) |
| N5 | C4 | C12 | C11 | 12 (2) | N55 | C54 | C62 | C61 | −2 (2) |
| N5 | C6 | C7 | C8 | −175 (3) | N55 | C56 | C57 | C58 | 177 (2) |
| C11 | C6 | C7 | C8 | 0.0 | C61 | C56 | C57 | C58 | 0.0 |
| C6 | C11 | C10 | C9 | 0.0 | C56 | C61 | C60 | C59 | 0.0 |
| C6 | C7 | C8 | C9 | 0.0 | C56 | C57 | C58 | C59 | 0.0 |
| C7 | C8 | C9 | C10 | 0.0 | C57 | C58 | C59 | C60 | 0.0 |
| C8 | C9 | C10 | C11 | 0.0 | C58 | C59 | C60 | C61 | 0.0 |
| C10 | C11 | C6 | N5 | 176 (2) | C60 | C61 | C56 | N55 | −177 (2) |
| C10 | C11 | C6 | C7 | 0.0 | C60 | C61 | C56 | C57 | 0.0 |
| C13 | C3 | C4 | C12 | −170 (4) | C63 | C53 | C54 | C62 | −171 (2) |
| C13 | C3 | C4 | N5 | −12 (4) | C63 | C53 | C54 | N55 | 12 (3) |
| C13 | C14 | C15 | C16 | −177 (4) | C63 | C64 | C65 | C66 | −176 (2) |
| C13 | C14 | C19 | C18 | 178 (4) | C63 | C64 | C69 | C68 | 175 (2) |
| C14 | C15 | C16 | C17 | 0.0 | C64 | C65 | C66 | C67 | 0.0 |
| C15 | C14 | C19 | C18 | 0.0 | C65 | C64 | C69 | C68 | 0.0 |
| C15 | C16 | C17 | C18 | 0.0 | C65 | C66 | C67 | C68 | 0.0 |
| C15 | C16 | C17 | 020 | −167 (5) | C65 | C66 | C67 | 070 | −159 (3) |
| C16 | C17 | C18 | C19 | 0.0 | C66 | C67 | C68 | C69 | 0.0 |
| C17 | C18 | C19 | C14 | 0.0 | C67 | C68 | C69 | C64 | 0.0 |
| C19 | C14 | C15 | C16 | 0.0 | C69 | C64 | C65 | C66 | 0.0 |
| 020 | C17 | C18 | C19 | 165 (5) | 070 | C67 | C68 | C69 | 160 (3) |
| 021 | C2 | C3 | C4 | 172 (3) | 071 | C52 | C53 | C54 | −168.4 (18) |
| 021 | C2 | C3 | C13 | −17 (4) | 071 | C52 | C53 | C63 | 5 (2) |
| C22 | C1 | C2 | C3 | −179 (2) | C72 | C51 | C52 | C53 | 178.8 (19) |
| C22 | C1 | C2 | 021 | 9 (4) | C72 | C51 | C52 | 071 | −11 (2) |
| C22 | C1 | C12 | C4 | 179 (2) | C72 | C51 | C62 | C54 | −179 (2) |
| C22 | C1 | C12 | C11 | 24 (6) | C72 | C51 | C62 | C61 | 2 (3) |
| C22 | C23 | C24 | C25 | 0.0 | C72 | C73 | C74 | C75 | 0.0 |
| C22 | C23 | C24 | C34 | 175 (2) | C72 | C73 | C74 | C84 | 172.0 (19) |
| C22 | C33 | C32 | C27 | 177 (2) | C72 | C83 | C82 | C77 | 172 (2) |
| C22 | C33 | C32 | C31 | −3 (4) | C72 | C83 | C82 | C81 | −15 (3) |
| C23 | C22 | C33 | C25 | 0.0 | C73 | C72 | C83 | C75 | 0.0 |
| C23 | C22 | C33 | C32 | −173 (3) | C73 | C72 | C83 | C82 | −175 (3) |
| C23 | C24 | C25 | C33 | 0.0 | C73 | C74 | C75 | C83 | 0.0 |
| C23 | C24 | C25 | N26 | 170 (5) | C73 | C74 | C75 | N76 | 166 (2) |
| C23 | C24 | C34 | C35 | 175 (2) | C73 | C74 | C84 | C85 | −179 (2) |
| C24 | C25 | C33 | C22 | 0.0 | C74 | C75 | C83 | C72 | 0.0 |
| C24 | C25 | C33 | C32 | 176 (2) | C74 | C75 | C83 | C82 | 177.5 (15) |
| C24 | C25 | N26 | C27 | −177 (2) | C74 | C75 | N76 | C77 | −174.2 (15) |
| C24 | C34 | C35 | C36 | −169 (2) | C74 | C84 | C85 | C86 | −172.5 (19) |
| C24 | C34 | C35 | C40 | 6 (3) | C74 | C84 | C85 | C90 | 11 (4) |
| C25 | C24 | C34 | C35 | −11 (4) | C75 | C74 | C84 | C85 | −10 (4) |
| C25 | C33 | C32 | C27 | 4.0 (17) | C75 | C83 | C82 | C77 | −3.6 (14) |
| C25 | C33 | C32 | C31 | −176.2 (16) | C75 | C83 | C82 | C81 | 169.7 (15) |
| C25 | N26 | C27 | C32 | 9 (4) | C75 | N76 | C77 | C82 | 6.2 (18) |
| C25 | N26 | C27 | C28 | 179.5 (19) | C75 | N76 | C77 | C78 | −169.9 (13) |
| C33 | C22 | C23 | C24 | 0.0 | C83 | C72 | C73 | C74 | 0.0 |
| C33 | C22 | C23 | 042 | 161 (3) | C83 | C72 | C73 | 092 | −168.4 (19) |
| C33 | C25 | N26 | C27 | −6 (4) | C83 | C75 | N76 | C77 | −9 (2) |
| C33 | C32 | C27 | N26 | −8 (3) | C83 | C82 | C77 | N76 | −1.5 (15) |
| C33 | C32 | C27 | C28 | 179.8 (18) | C83 | C82 | C77 | C78 | 174.7 (14) |
| C33 | C32 | C31 | C30 | −180 (3) | C83 | C82 | C81 | C80 | −172 (2) |
| N26 | C25 | C33 | C22 | −174 (3) | N76 | C75 | C83 | C72 | −169.2 (19) |
| N26 | C25 | C33 | C32 | 1 (3) | N76 | C75 | C83 | C82 | 8 (2) |
| N26 | C27 | C28 | C29 | −171 (3) | N76 | C77 | C78 | C79 | 176.0 (17) |
| C32 | C27 | C28 | C29 | 0.0 | C82 | C77 | C78 | C79 | 0.0 |
| C27 | C32 | C31 | C30 | 0.0 | C77 | C82 | C81 | C80 | 0.0 |
| C27 | C28 | C29 | C30 | 0.0 | C77 | C78 | C79 | C80 | 0.0 |
| C28 | C29 | C30 | C31 | 0.0 | C78 | C79 | C80 | C81 | 0.0 |
| C29 | C30 | C31 | C32 | 0.0 | C79 | C80 | C81 | C82 | 0.0 |
| C31 | C32 | C27 | N26 | 172 (3) | C81 | C82 | C77 | N76 | −176.2 (16) |
| C31 | C32 | C27 | C28 | 0.0 | C81 | C82 | C77 | C78 | 0.0 |
| C34 | C24 | C25 | C33 | −175 (2) | C84 | C74 | C75 | C83 | −170 (2) |
| C34 | C24 | C25 | N26 | −4 (5) | C84 | C74 | C75 | N76 | −3 (3) |
| C34 | C35 | C36 | C37 | 176 (2) | C84 | C85 | C86 | C87 | −177 (2) |
| C34 | C35 | C40 | C39 | −176 (2) | C84 | C85 | C90 | C89 | 176 (2) |
| C35 | C36 | C37 | C38 | 0.0 | C85 | C86 | C87 | C88 | 0.0 |
| C36 | C35 | C40 | C39 | 0.0 | C86 | C85 | C90 | C89 | 0.0 |
| C36 | C37 | C38 | C39 | 0.0 | C86 | C87 | C88 | C89 | 0.0 |
| C36 | C37 | C38 | 041 | 173 (2) | C86 | C87 | C88 | 091 | −171 (2) |
| C37 | C38 | C39 | C40 | 0.0 | C87 | C88 | C89 | C90 | 0.0 |
| C38 | C39 | C40 | C35 | 0.0 | C88 | C89 | C90 | C85 | 0.0 |
| C40 | C35 | C36 | C37 | 0.0 | C90 | C85 | C86 | C87 | 0.0 |
| 041 | C38 | C39 | C40 | −173 (2) | 091 | C88 | C89 | C90 | 170 (2) |
| 042 | C23 | C24 | C25 | −163 (2) | 092 | C73 | C74 | C75 | 168 (2) |
| 042 | C23 | C24 | C34 | 13 (2) | 092 | C73 | C74 | C84 | −20 (2) |

### Notes

It is noted that the crystal was found to be multiply twinned within the processing of collected diffraction data, and adequate procedures implemented by the software manufacturer had to be used. Structure model refinement parameters (R₁, wR₂, GoF) (Table 4) are far from satisfactory, but the fact that the original model obtained during phase problem solving agreed with expectations and was chemically consistent strongly suggested that the assumed structure model was correct. In addition, the model had relatively stable behavior during refinement, which means that no model disintegration occurred, even though this frequently occurs whenever a structure model does not correspond to reality.

The proposed structure model assumes lattice symmetry consistent with the Pc space group. The asymmetric unit contains two molecules of the analyzed compound (FIG. 8a).

Each of the molecules consists of two largely coplanar fragments. Therefore, torsion angles to a significant degree determine the conformation of the analyzed molecules: C2-C1-C22-C23 −142.9 (16)° and C52-C51-C72-C73 −144.8 (14)°, respectively.

The distance of 2.71 Å between the O20 and O70 terminal hydroxyl oxygen atoms suggests a hydrogen bond between the atoms.

Two C101 and C102 atoms not bound covalently are noted in the model, preliminarily classified as carbon atoms. These could be artifacts resulting from the quality of obtained data, but their distances from hydroxyl oxygen atoms (O41-C101 2.679 Å and O91-C102 2.734 Å) suggest that hydrogen bonds could be present in this location. It could therefore be supposed that these would be oxygen atoms found in residual solvent molecules.

### EXAMPLE 9

A scytonemin sample obtained according to Example 1 was dissolved in DMSO (dimethylsulfoxide) to a concentration of 1% by weight and subsequently, with a spectrophotometer used according to the standard procedures (manufacturer: Varian, model: CARY 100 Scan) absorbance was measured for two wavelengths (305 and 393 nm) in a cuvette with 1 cm thickness. The following results were obtained:

UV absorption and extinction coefficients:
Specific extinction/1% at 305 nm: 330
Specific extinction/1% to 393 nm: 730

### REFERENCES

Proteau PJ, Gerwick Garcia-Pichel F, Castenholz R. Experientia 1993, 49, 825-829.
Fleming ED, Castenholz RW. 2007. Effects of periodic desiccation on the synthesis of the UV-screening compound, scytonemin, in Cyanobacteria. Environ Microbiol 9: 1448-1455.
Rastogi RP, Incharoensakdi A. 2014. Characterization of UV-screening compounds, mycosporine-like amino acids, and scytonemin in the Cyanobacterium lyngbya sp. CU2555. FEMS Microbiology Ecology 87: 244-256.
Wolk CP. (1988) Purification and storage of nitrogen fixing filamentous Cyanobacteria
Methods Enzymol., 167, pp. 93-100
Anahas AMP, Muralitharan G (2015) Isolation and screening of heterocystous cyanobacterial strains for biodiesel production by evaluating the fuel properties from fatty acid methyl ester (FAME) profiles. Bioresource technology 184:9-17. doi:https://doi.org/10.1016/j.biortech.2014.11.003
Balskus EP, Case RJ, Walsh CT (2011) The biosynthesis of cyanobacterial sunscreen scytonemin in intertidal microbial mat communities. FEMS Microbiol Ecol 77 (2):322-332. doi:10.1111/j.1574-6941.2011.01113.x
Rippka R, Deruelles J, Waterbury JB (1979) Generic assignments, strain histories and properties of pure cultures of Cyanobacteria. Journal of General Microbiology 111 (1):1-61
Singh J, Tripathi R, Thakur IS (2014) Characterization of endolithic cyanobacterial strain, Leptolyngbya sp. ISTCY101, for prospective recycling of CO2 and biodiesel production. Bioresource technology 166:345-352. doi:https://doi.org/10.1016/j.biortech.2014.05.055
Greiter F, (1974) Sun protection factor-development methods. Parf Kosm. 55:70-75
Rigaku Oxford Diffraction 2018, CrysAlisPro Software system, version 1.171.64.93a, Rigaku Corporation, Oxford, UK.
Sheldrick GM, SHELXT-Integrated Space-Group and Crystal-Structure Determination, Acta Crystallographica Section A 2015, A71, 3-8.
Sheldrick GM, Crystal structure refinement with SHELXL Acta Crystallographica, Section C 2015, C71, 3-8.
Macrae CF, Sovago I, Cottrell SJ, Galek PTA, McCabe P, Pidcock E, Platings M, Shields GP, Stevens JS, Towler M and Wood PA, Mercury 4.0: from visualization to analysis, design and prediction, J. Appl. Cryst. 2020, 53, 226-235.

1. A process for the isolation and culture of Cyanobacteria strains, in particular that deposited in Banco Espanol de Algas Universidad de Las Palmas de GC under number BEA_IDA_0068B or BEA_IDA_0075B, characterized in that it comprises:
a) preparation of a growth medium by enriching it in micro- and macronutrients found in natural sandstone originating from Nubian formations with the following contents in mass percentages: 97.6% quartz, 0.4% muscovite-biotite 1.2% apatite and 0.8% other minerals in trace quantities in the amount of 200 g per 1000 mL of an aqueous medium solution having the following composition per 1000 ml of the medium: 1.5 g NaNO₃, 0.04 g K₂HPO₄, 0.075 g MgSO₄×7H₂O, 0.036 g CaCl₂×2H₂O, 6.0 mg citric acid, 6.0 mg ammonium ferric citrate, 1 mg EDTA, 0.02 g Na₂CO₃, 1 mL of the A5 blend of trace metals with the following composition per 1000 mL of the aqueous A5 blend solution: 2.86 g H₃BO₃, 1.81 g MnCl₂×4H₂O, 0.222 g ZnSO₄×7H₂O, 0.39 g Na₂MoO₄×2H₂O, 0.079 g CuSO₄×5H₂O, 49.4 mg Co (NO₃)₂×6H₂O, subsequently stirring the resulting suspension for 24 hours at 25° C. and subsequent 5-hour sedimentation at 25° C. and filtration thereof;
b) collection of bacteria from the environment;
c) passaging the biological material collected in stage b) in the liquid medium obtained in stage a), i.e., according to Table 1, enriched with stone, with additional agar with end contents between 2% in the beginning and 0.5% by weight in the end, preferably in three intermediate stages of 4 weeks each of the five stages, i.e. two end stages (initial, final) and three intermediate stages, wherein the growth media in the intermediate stages contain the following quantities of additional agar: 1.75%, 1.5%, 1% by weight, respectively, with respect to the medium obtained in stage a);
d) dissolving the culture solution from final stage c), i.e. containing 0.5% agar, in the aqueous medium solution whose composition is disclosed in stage A but without addition of the stone and incubation at 25° C. for 2 weeks with stirring.

2. A new bacterial strain deposited in Banco Espanol de Algas Universidad de Las Palmas de GC under number BEA_IDA_0068B.

3. A new bacterial strain deposited in Banco Espanol de Algas Universidad de Las Palmas de GC under number BEA_IDA_0075B.

4. Use of the strain as defined in claim 2 for the manufacture of a pigment having UV absorption properties, in particular scytonemin or derivatives thereof.

5. The use of claim 4 comprises application of the resulting pigment, in particular scytonemin or derivatives thereof, for the manufacture of cosmetic products, in particular for sunscreens.

6. A medium for culturing Cyanobacteria, containing in 1000 mL of the aqueous medium solution 1.5 g NaNO₃, 0.04 g K₂HPO₄, 0.075 g MgSO₄×7H₂O, 0.036 g CaCl₂×2H₂O, 6.0 mg citric acid, 6.0 mg ammonium ferric citrate, 1 mg EDTA, 0.02 g Na₂CO₃, 1 mL of the A5 blend of trace metals with the following composition per 1000 mL of the aqueous A5 blend solution: 2.86 g H₃BO₃, 1.81 g MnCl₂×4H₂O, 0.222 g ZnSO₄×7H₂O, 0.39 g Na₂MoO₄×2H₂O, 0.079 g CuSO₄×5H₂O, 49.4 mg Co (NO₃)₂×6H₂O, characterized in that it contains natural Nubian sandstone with the following contents in mass percentages: 97.6% quartz, 0.4% muscovite-biotite 1.2% apatite and 0.8% other minerals in trace quantities in the amount of 200 g ground stone/1000 mL of the medium.

7. Scytonemin crystals having at least one property selected from the following:
X-ray powder diffraction spectrum with characteristic peaks at 2 theta angle values of 2.500°, 4.589°, 5.062°, 8.630° and 9.197°,
specific infrared absorption bands at 3345, 3065, 2961, 2926, 1713, 1591, 1516, 1449, 1296, 1175, 1145, 957, 932, 930, 833 [cm⁻¹] in the IR spectrum (KBr),
decomposition temperature in a range between 365° C. and 380.3° C. with a peak at about 380.3° C. in thermogravimetric/differential thermal analysis (heating/cooling rate: 15/20° C./min).
¹H NMR spectrum recorded in pyridine-d₅ containing signals at δ 8.98 ppm; 7.99 ppm; 7.86 ppm; 7.75 ppm; 7.48 ppm; 7.33 ppm; 7.22 ppm.
structure model based on structural X-ray analysis (XRD) described by the geometric parameters listed in Tables 4 to 7 and presented in FIGS. 8a to d.
specific extinction 1% in DMSO at 393 nm: 730 and specific extinction 1% in DMSO at 305 nm: 330.

8. Use of the strain as defined in claim 3 for the manufacture of a pigment having UV absorption properties, in particular scytonemin or derivatives thereof.

9. Use of the strain as defined in claim 3 for the manufacture of a pigment having UV absorption properties, in particular scytonemin or derivatives thereof.

10. The use of claim 5 comprises application of the resulting pigment, in particular scytonemin or derivatives thereof, for the manufacture of cosmetic products, in particular for sunscreens.
